(19) United States
(12) Patent Application Publication
(10) Pub. No.: US 2013/0324947 A1
(43) Pub. Date: Dec. 5, 2013

(54) TENSION RELIEF BAG

(71) Applicant: Janice Ann Rigmaiden, Castle Pines, CO (US)

(72) Inventor: Janice Ann Rigmaiden, Castle Pines, CO (US)

(21) Appl. No.: 13/481,957

(22) Filed: May 29, 2012

(51) Int. Cl. A61M 35/00

(52) U.S. Cl. 604/289

(57) ABSTRACT

The present invention is a tension relief bag that includes a plurality of washing soap intended to be used by a human, a two-sided cloth containing pouch with a tucked in flap that contains the washing soap and a flap that is disposed of the containing pouch. The tension relief bag can utilize washing soap that is a plurality of shredded soap or one or more bars of washing soap. The tension relief bag flap can be tucked into the containing pouch or be releasable secured to the containing pouch with a tucked in flap.

### TECHNICAL FIELD & BACKGROUND

It is well known that many individuals have trouble sleeping due to muscular contractions. Currently there are limited alternatives to natural remedies which mitigate such ailments.

The present invention generally relates to a bag. More specifically, the invention is a tension relief bag.

It is an object of the invention to provide a tension relief bag that is in contact with a human body that prevents or relieves muscle tension and spasms.

It is an object of the invention to provide a tension relief bag that utilizes a plurality of soap pieces, a cloth bag and a closable flap.

It is an object of the invention to provide a tension relief bag that is in the bottom of individuals mattress.

What is really needed is a tension relief bag that is in contact with a human body that prevents or relieves muscle tension and spasms that utilizes a plurality of soap pieces, a cloth bag and a closable flap that is in the bottom of individuals' mattress.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a side view of a tension relief bag, according to an embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is utilized repeatedly. The phrase generally does not refer to the same embodiment, however, it may. The terms “comprising”, “having” and “including” are synonymous, unless the context dictates otherwise.

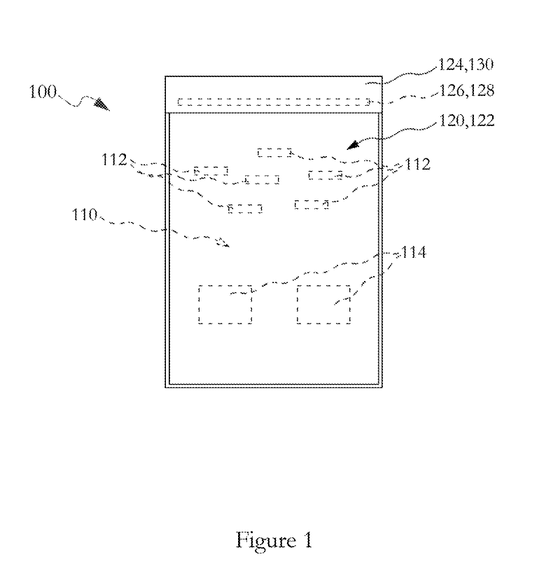

FIG. 1 illustrates a side view of a tension relief bag 100, according to an embodiment of the present invention.

The tension relief bag 100 includes a plurality of washing soap 110, a containing pouch 120 and a flap 130. The washing soap 110 can be any washing soap intended to be used by a human that is shredded into a plurality of shreds 112 that can be any suitable sized shreds or can be one or more bars 114 of washing soap intended to be used by a human. The containing pouch 120 is a two-sided pouch 122 with a tucked in flap 124 that is made of cloth or other suitable material. The containing pouch 120 contains the washing soap 110. The containing pouch 120 contains the flap 130 that is disposed the containing pouch 120. The flap 130 can be tucked into the containing pouch 120 or releasably secured to the containing pouch 130 with a suitable tucked in sealer 126 in a strip form 128 or other suitable fastener.

The tension relief bag 100 is a therapy designed to be placed in between an individual's mattress and a bottom sheet (not shown). The tension relief bag 100 has dimensions of approximately 9 inches×18 inches or approximately 6 inches×6 inches.

The plurality of shredded soap is inserted into a decorative pouch and is slipped underneath a patient's bed, only requiring the patient to lie down to procure treatment. In order to prevent the soap contents from disbursing throughout the mattress, simply tuck the provided flap inside the bag after inserting the shredded soap. The tension relief bag may be readily available wherever pain relief products are sold in various sizes, colors and designs. The tension relief bag is able to naturally ease pain or discomfort due to muscle spasms of a person's lower extremities and may be also used when at the office or when traveling in a car. The tension relief bag features an aesthetically fashionable pouch, which contains a shredded soap filling and provides a secure seal to prevent the shredded soap from spilling.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A tension relief bag, comprising:
a plurality of discrete washing soap pieces;
a containing pouch that contains said washing soap pieces, wherein said containing pouch is configured to be placed between a conventional bed mattress and a conventional bed sheet so as to allow a user to lay on it in a flat position to obtain treatment from the soap pieces in the containing pouch; and
a flap that is disposed on said containing pouch, wherein said flap is adapted to be tucked into said containing pouch.

2. The tension relief bag according to claim 1, wherein said washing soap is a plurality of shredded soap.

3. The tension relief bag according to claim 1, wherein said washing soap is one or more bars of said washing soap.

4. The tension relief bag according to claim 1, wherein said containing pouch is a two-sided pouch.

5. The tension relief bag according to claim 1, wherein said containing pouch is made of cloth.

6. The tension relief bag according to claim 1, wherein said flap is tucked into said containing pouch.

7. The tension relief bag according to claim 1, wherein said flap is releasable secured to said containing pouch with tucked in flap.

8. The tension relief bag according to claim 7, wherein a flap that is sealed.

9. The tension relief bag according to claim 1, wherein said tension relief bag is placed on between the bottom of an individual's mattress and a bottom sheet.

10. (canceled)

11. A tension relief bag, comprising:
a plurality of discrete washing soap pieces;
a two-sided cloth containing pouch with a top portion that contains said washing soap pieces, wherein said containing pouch is configured to be placed between a conventional bed mattress and a conventional bed sheet so as to allow a user to lay on it in a flat position to obtain treatment from the soap pieces in the containing pouch; and
a flap that is disposed on said containing pouch, wherein said flap is adapted to be tucked into said containing pouch.

12. The tension relief bag according to claim 11, wherein said washing soap is a plurality of shredded soap.

13. The tension relief bag according to claim 11, wherein said washing soap is one or more bars of said washing soap.

14. The tension relief bag according to claim 11, wherein said flap is tucked into said containing pouch.

15. The tension relief bag according to claim 11, wherein said flap is releasable secured to said containing pouch with a tucked in flap.

16. The tension relief bag according to claim 15, wherein said flap is tucked.

17. The tension relief bag according to claim 11, wherein said tension relief bag is placed on the bottom of an individual's mattress.

18. The tension relief bag according to claim 11, wherein said tension relief bag has dimensions of approximately 9 inches×18 inches.

19. The tension relief bag according to claim 11, wherein said tension relief bag has dimensions of approximately 6 inches×6 inches.

20. A method of preventing or relieving muscle tension and spasms, comprising the steps of:
providing a plurality of discrete washing soap pieces;
providing a two-sided cloth containing pouch with said plurality of discrete washing soap pieces disposed inside, wherein said containing pouch includes a flap which is adapted to be tucked into said containing pouch; and
placing said containing pouch between a conventional bed mattress and a conventional bed sheet such that it is configured to be laid on by a user in a manner which would allow the containing pouch to contact the user's body through the sheet with the user being in a flat position.

21. The method of claim 21, wherein the plurality of discrete washing soap pieces are defined by a plurality of shredded soap pieces.
